(19) United States
(12) Patent Application Publication
(10) Pub. No.: US 2006/0223165 A1
(43) Pub. Date: Oct. 5, 2006

(54) Device for counting cells and method for manufacturing the same

(75) Inventors: Jun Keun Chang, Seoul (KR); Seok Chung, Seoul (KR); Chanil Chung, Kyunggi-Do (KR); Hyun Woo Bang, Seoul (KR); Han Sang Jo, Kyunggi-Do (KR)

Correspondence Address: BROWDY AND NEIMARK, P.L.L.C.;624 NINTH STREET, NW, SUITE 300, WASHINGTON, DC, 20001-5303, US

(73) Assignee: Digital Bio Technology, Seoul (KR)

(21) Appl. No.: 10/565,079

(22) PCT Filed: Jul. 13, 2004
(86) PCT No.: PCT/KR04/01735

§ 371 Date: Jan. 18, 2006

(30) Foreign Application Priority Data
Jul. 18, 2003 (KR) 10 2003 0049406

(51) Int. Cl. C12M 1/34; B44C 3/08

(52) U.S. Cl. 435/287.1; 156/219

(57) ABSTRACT

Disclosed are a device for counting cells and a method for manufacturing the device. The device comprises a transparent upper substrate and a transparent lower substrate. Fine lattice patterns for counting cells are formed on the lower substrate. A space for filling the sample, including cells, is provided between the upper substrate and the lower substrate. A hole for injecting the samples into the space for filling the samples is provided in the upper substrate. The device is disposable, and the cost of manufacturing the device is low. It is easy to fill the samples into the filling space and to count cells by using the device. The upper substrate and the lower substrate are bonded, and thus, no cover is required.

### TECHNICAL FIELD

The present invention relates to a device for counting cells, and in particular to a device for counting cells comprising a transparent lower substrate having fine lattice patterns for counting the cells formed on an upper surface thereof and a transparent upper substrate stacked on the lower substrate, wherein the upper substrate comprises a fill chamber having a predetermined height from a bottom surface of the upper substrate and forming a space for filling a sample including the cells into the fine lattice patterns and an injecting hole for the sample communicated with the fill chamber.

### BACKGROUND ART

Generally, when diagnosing a disease, it is examined the number and the functions of typical blood cells such as erythrocytes, leukocytes or platelets included in the blood. For example, it is possible to diagnose tuberculosis, obesity or pregnancy from a blood sedimentation rate and dehydration or anemia from a corpuscular volume. Also, it is possible to diagnose chronic leukemia from the number of platelets, kidney disease, hypoxia, smoking, pulmonary disease, hemolytic anemia or aplastic anemia from the number of erythrocytes, and acute typhlitis, leukemia or aplastic anemia from the number of leukocytes. Like this, the measurement of the number of blood cells is closely related to the disease diagnosis.

The size of erythrocytes, which are typical blood cells, is classified into micro, normal, macro and mega. By finding out the sizes and the number of erythrocytes, it is possible to use them as diagnostic materials for various diseases as described above.

Particularly, it is required to know the number of erythrocytes for seeking whether or not anemia and cause thereof. For the healthy public, a male has about 4,400,000˜5,600,000 erythrocytes/dl in blood and a female has about 3,500,000˜5,000,000 erythrocytes/dl.

From the measurement of the number of erythrocytes, when the number of erythrocytes is increased beyond a reference value, it is possible to diagnose diseases such as polycythemia vera, dehydration, shock adrenal insufficiency or cardiopulmonary disease. Also, when the number of erythrocytes is decreased, it is possible to diagnose whether or not various anemia.

FIG. 1 is a perspective view showing a device for measuring the number of blood cells such as erythrocytes according to the prior art.

As shown in FIG. 1, the device 10 for measuring the number of erythrocytes according to the prior art comprises a body 15 made of glass or quartz, partition walls 20, 25 provided on an upper part of the body 15, a measurement part 30 formed between the partition walls 20, 25 and a cover 35 covering an upper part of the measurement part 30.

The partition walls 20, 25 located on the body 15 and the measurement part 30 located between the partition walls 20, 25 are formed on the body 15 by micromachining the body 15 made of glass or quartz as a method disclosed in Korean Unexamined Patent Publication No. 1999-84670, for example.

The partition walls 20, 25 are upwardly protruded from the upper part of the body 15 at the periphery of the measurement part 30 so that a sample such as blood is not flown out of the measurement part 30 when the sample is poured into the measurement part 30. Also, the cover 35 made of glass is provided on the partition walls 20,25, so that the sample is in existence in the measurement part 30 between the partition walls 20, 25 and the cover 35 and thus the number of cells in the sample, such as blood cells in the blood, is measured.

FIG. 2 is a schematic plan view showing the measurement part of the device shown in FIG. 1.

As shown in FIGS. 1 and 2, the measurement part 30 consists of a plurality of measuring areas 45 and bright lines 40 for distinguishing each of the measuring areas 45.

Generally, the bright lines 40 are arranged in a cross pattern and thus divide the measurement part 30 into four measuring areas 45. A plurality of lattice lines, which are arranged lengthwise and crosswise according to the sizes of cells to be measured, are formed in each measuring area 45. For example, blood is dropped in the measuring areas 45 having such a construction and thus the number of cells, which are in existence between the lattice lines, is measured.

However, in the device for measuring the number of cells such as erythrocytes, since the body made of glass or quartz is relatively expensive and it takes much time and efforts to micromachine the body, the time and cost for manufacturing the device are increased. Like this, since the prior device for measuring the number of cells is expensive, it is required that once the device is used, it should be washed and then reused. Accordingly, it should be put up with inconveniences of washing the device and there is a possibility that the sample previously measured remains in the device.

Also, since the device made of glass or quartz is fragile by an impact, there is some danger that the device is damaged during using it.

In addition, since the body 15 and the cover 35 are separated, there is troublesomeness that the sample should be dropped after covering the body with the cover. Particularly, since the tightness between the cover and the partition walls is very poor, it is required that a very strong force should be applied to the cover or the cover should be attached to the partition walls with a separate adhesive. Also, there is a possibility that the cover is damaged during using the device even if such an additional process was used.

### DISCLOSURE OF INVENTION

Accordingly, the present invention has been made to solve the above-mentioned problems occurring in the prior art. The object of the present invention is to provide a device for counting cells. The other object of the invention is to provide a manufacturing method of the device.

In order to accomplish the objects, the invention comprises a transparent lower substrate having fine lattice patterns for counting cells formed on an upper surface thereof; and a transparent upper substrate stacked on the lower substrate, wherein the upper substrate comprises a fill chamber having a predetermined height from a bottom surface of the upper substrate and forming a space for filling a sample including the cells on the fine lattice patterns and an injecting hole for the sample communicated with the fill chamber.

When the upper substrate and the lower substrate are integratedly made by bonding them, a covering process as the prior device for counting cells is not required. Accordingly, since it is easy to fill the fill chamber with the sample by dropping the sample into the injection hole, the device can be more easily used than that of the prior art. Also, since the cost of manufacturing the device for counting cells can be greatly decreased, the device can be disposably and easily used.

The invention relates to a device for counting fine particles such as cells.

More specifically, the invention provides a device for counting fine particles comprising a transparent lower substrate having fine lattice patterns for counting the fine particles formed on an upper surface thereof; and a transparent upper substrate stacked on the lower substrate, wherein the upper substrate comprises a fill chamber having a predetermined height from a bottom surface of the upper substrate and forming a space for filling a sample including the fine particles on the fine lattice patterns and an injecting hole for the sample communicated with the fill chamber.

Preferably, the upper substrate further comprises a discharge hole communicated with the fill chamber for discharging the sample or an air bubble from the fill chamber.

Particularly, the upper and lower substrates are preferably bonded and thus form an integrated body. The upper and lower substrates are bonded by a convenient method such as a heating, an adhesive, a coating, a pressurization or a vibration, preferably an ultrasonic bonding.

According to the invention, a height of the fill chamber may be arbitrarily formed according to a volume of the sample to be examined. The height is preferably 50-200 μm and most preferably 100 μm.

According to the invention, an area of the fill chamber in the upper and lower substrates is made to be transparent for a microscopic observation. The fine lattice patterns are formed in a predetermined place of the area in which the fill chamber is formed on the lower substrate. It is possible to calculate a volume of the fill chamber by making the area of the fill chamber have a predetermined area and a predetermined height.

An indicative member is preferably formed on the upper substrate for indicating a position of the fine lattice patterns. Accordingly, when counting the cells in the sample with a microscope, it is possible to easily find the position of the fine lattice patterns.

The upper substrate or lower substrate may be made by an arbitrary material, referably any plastics capable of being injection-molded such as polycarbonate (PC), olymethylmethacrylate (PMMA), polyethylene (PE), polyethyleneterephthalate PET) or polystyrol (PS).

By using the device according to the invention, it is possible to easily count rythrocytes, leukocytes or platelets included in the blood. Additionally, it is possible o easily count microbes which are unicellular organisms, bacteria and any fine articles.

In addition, the invention provides a manufacturing method of a device for counting fine particles comprising steps of forming fine lattice patterns on a redetermined place of a lower substrate; forming a fill chamber having a predetermined height for filling a sample including the fine particles, such as blood cells or bacteria, an injecting hole and a discharge hole communicated with the fill chamber in an upper substrate; and bonding the upper and lower substrates.

### BRIEF DESCRIPTION OF DRAWINGS

The above and other objects, features and advantages of the present invention will be more apparent from the following detailed description taken in conjunction with the accompanying drawings, in which:

FIG. 1 is a perspective view showing a device for measuring the number of erythrocytes according to the prior art;

FIG. 2 is a schematic plan view showing a measurement part of the device shown in FIG. 1;

FIG. 3 is a perspective view of an upper substrate of a device according to an embodiment of the invention;

FIG. 4 is a sectional view of the upper substrate shown in FIG. 3;

FIG. 5 is a plan view of the upper substrate shown in FIG. 3;

FIG. 6 is a perspective view of a lower substrate of the device according to an embodiment of the invention;

FIG. 7 shows fine lattice patterns formed on the lower substrate;

FIG. 8 shows an embodiment of the invention, in which upper and lower substrates are bonded;

FIG. 9 shows another embodiment of the invention;

FIG. 10a to 10d are sectional views for illustrating an example of a process of forming fine lattice patterns on the lower substrate;

FIG. 11a to 11h are sectional views for illustrating another example of a process of forming fine lattice patterns on the lower substrate.

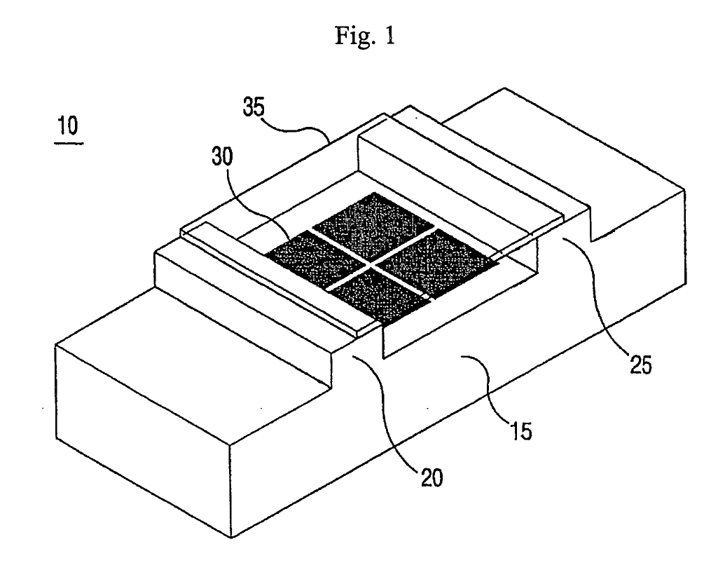

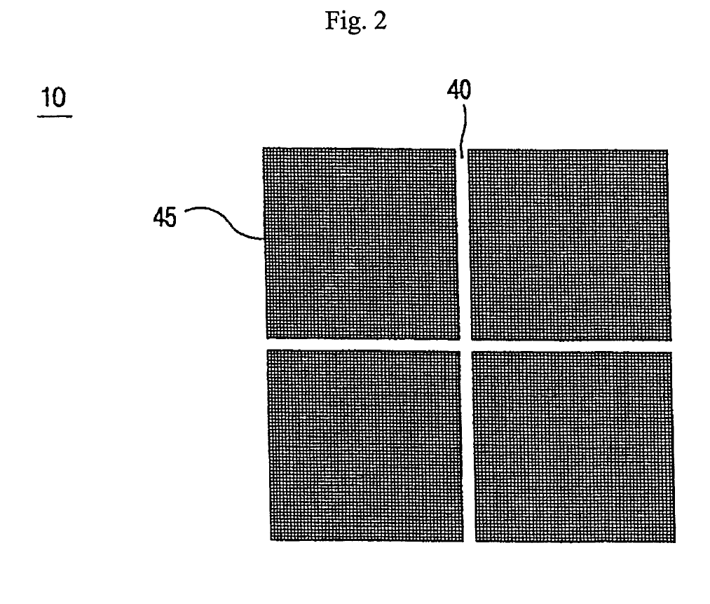

### DESCRIPTION OF REFERENCE NUMERALS FOR IMPORTANT PART OF THE DRAWINGS

| | |
|---|---|
| 100: upper substrate | 110: fill chamber |
| 120: injecting hole | 130: discharge hole |
| 140: indicative member | 200: lower substrate |
| 210: fine lattice patterns | |

### BEST MODE FOR CARRYING OUT THE INVENTION

Hereinafter, preferred embodiments of the present invention will be described with reference to the accompanying drawings. In the following description of the present invention, a detailed description of known functions and configurations incorporated herein will be omitted when it may make the subject matter of the present invention rather unclear.

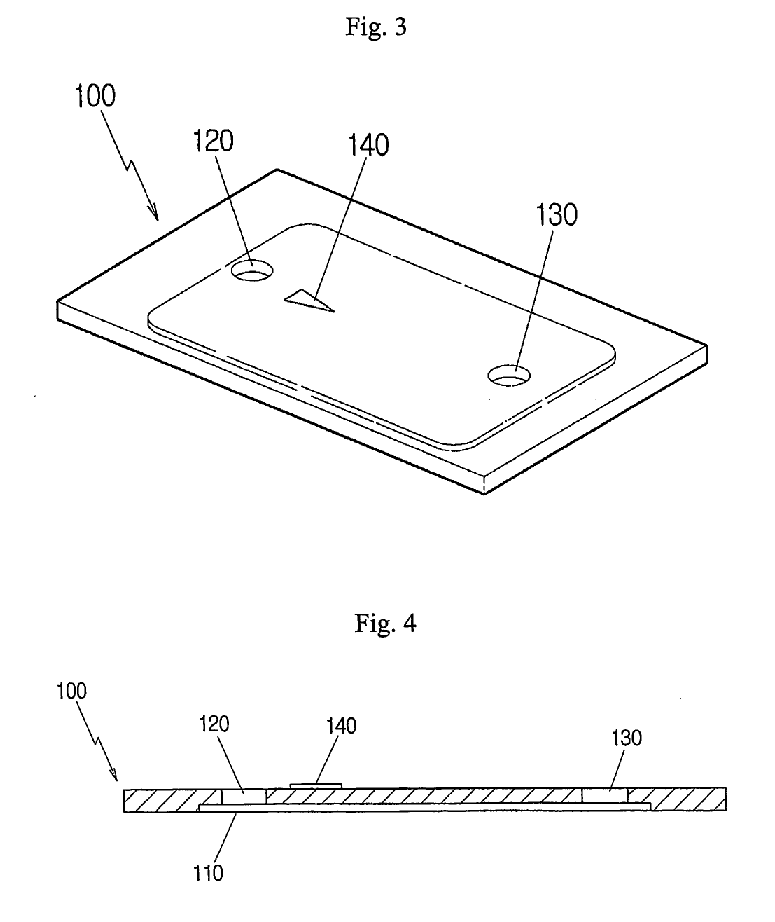

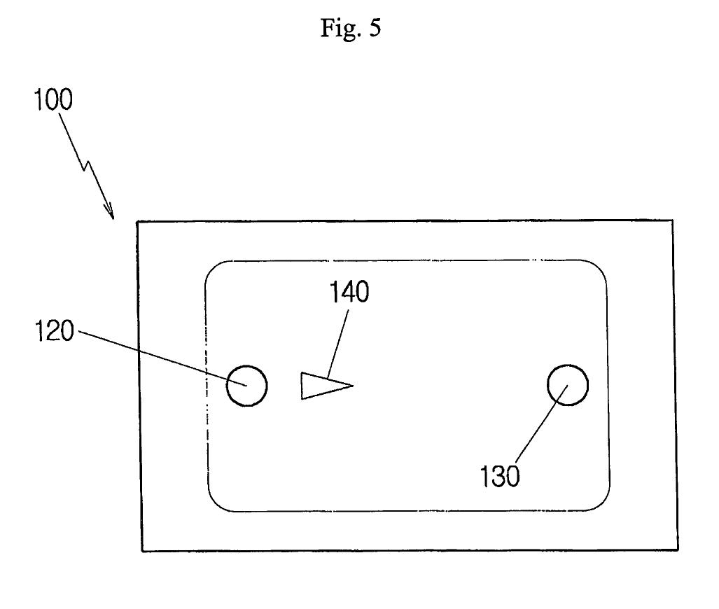

FIG. 3 is a perspective view showing an upper substrate of a device according to an embodiment of the invention, FIG. 4 is a sectional view of the upper substrate and FIG. 5 is a plan view of the upper substrate.

As shown in FIGS. 3 to 5, the upper substrate 100 comprises a fill chamber 110 having a predetermined height from a bottom surface of the upper substrate and forming a space for filling a sample, an injecting hole 120 for the sample communicated with the fill chamber and a discharge hole 130 for discharging the air and the excess sample in the fill chamber 110 when injecting the sample. In addition, an indicative member 140 is formed on the upper substrate 100 for indicating a position of fine lattice patterns formed on a lower substrate.

The sample can be easily injected when the injecting hole 120 and the discharge hole 130 are provided at opposite positions.

The upper substrate is made of transparent plastics and can be manufactured by a typical injection molding.

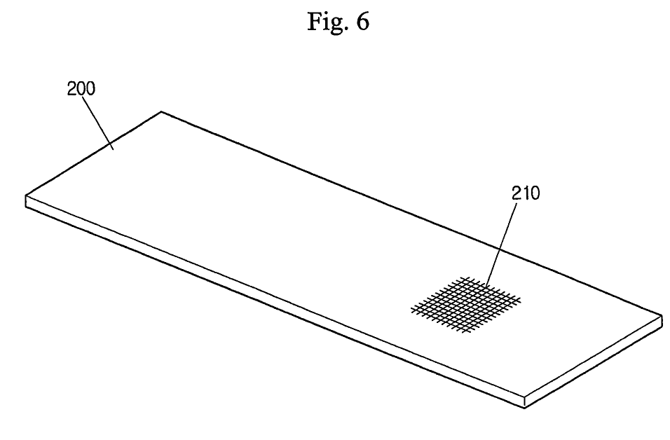

FIG. 6 is a perspective view showing a lower substrate of the device according to an embodiment of the invention. Transparent plastics capable of being injection-molded is used for the lower substrate.

Fine lattice patterns 210 for counting cells of the sample are formed on the lower substrate. The upper substrate is stacked on the lower substrate, so that the fill chamber 110 is formed. A method for forming the fine lattice patterns will be described later.

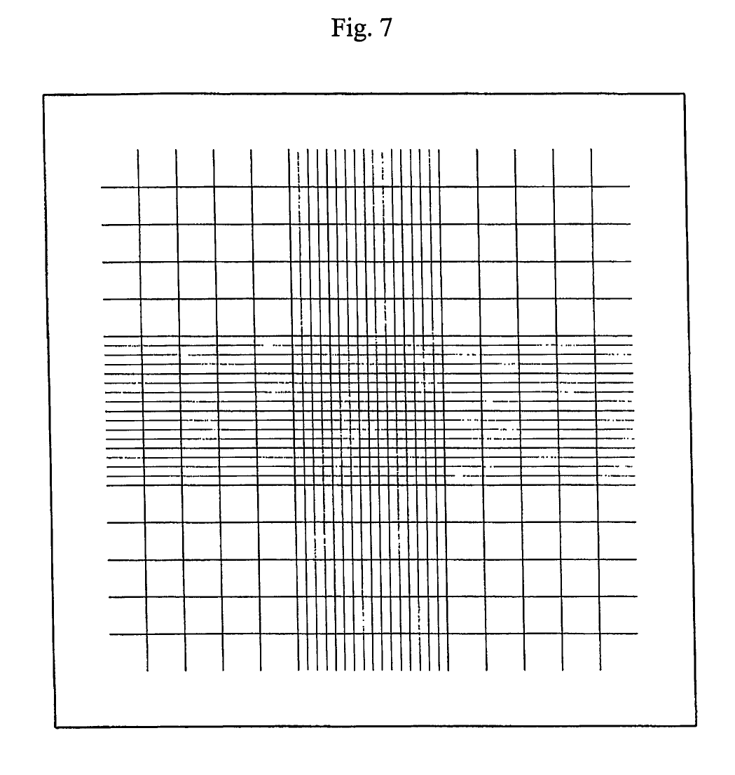

FIG. 7 is an enlarged view of the fine lattice patterns formed on the lower substrate. Shape, height, width and interval, etc. of the fine lattice patterns can be arbitrarily formed as necessary. Preferably, the height, the width and the interval of the fine lattice patterns are about 1 μm, about 1.5 μm and 10 μm, respectively.

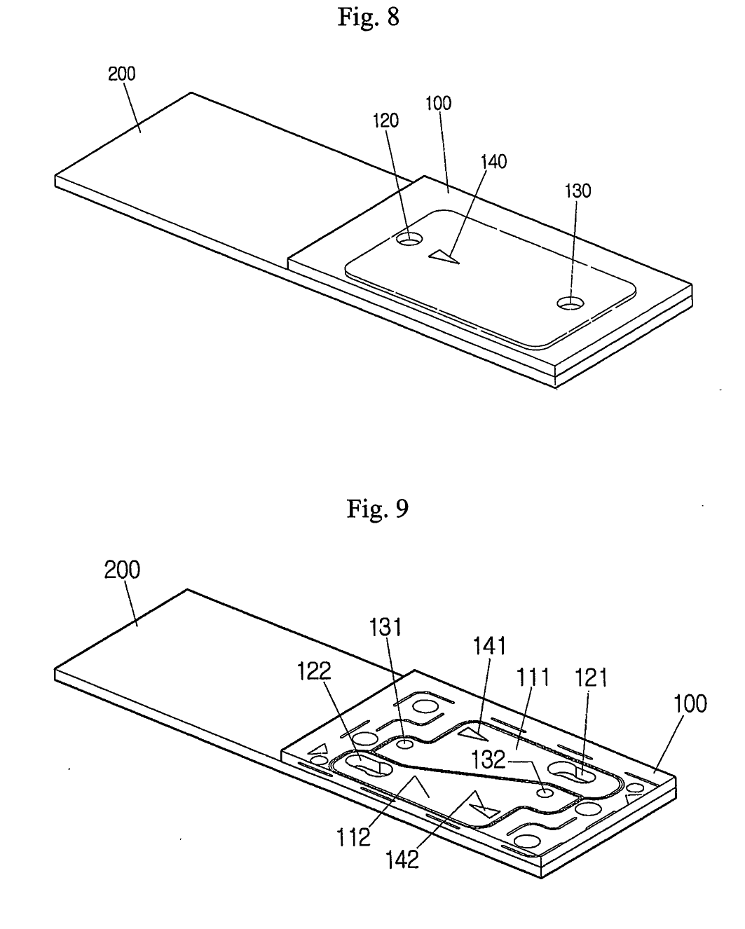

FIG. 8 shows a device according to an embodiment of the invention, which is integratedly made by bonding the upper and lower substrates with an ultrasonic bonding.

FIG. 9 shows a device according to another embodiment of the invention, which device comprises two fill chambers 111, 112 separated by a partition wall. Thus, injecting holes 121, 122, discharge holes 131, 132 and indicative members 141, 142 are separately formed in each of the fill chambers 111, 112. As shown in FIG. 9, the device may comprise at least two fill chambers as necessary.

Hereinafter, a method for forming fine lattice patterns on the lower substrate will be described.

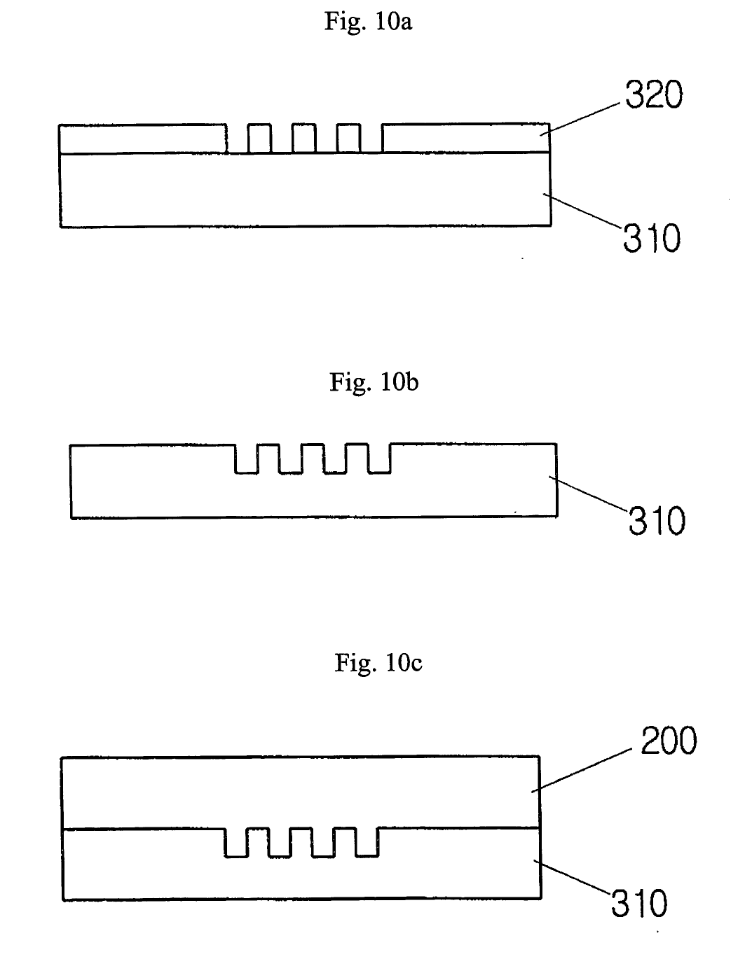

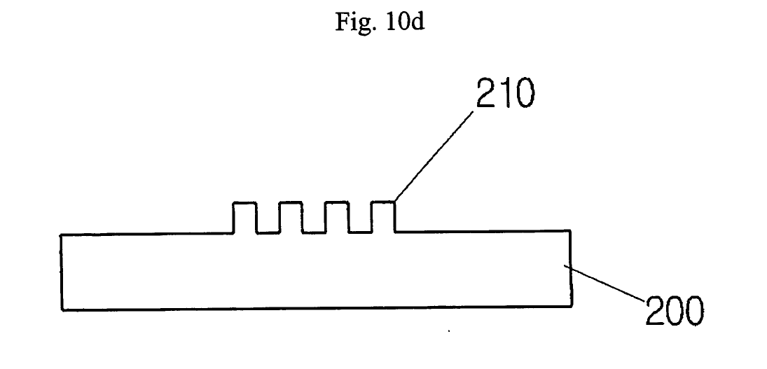

FIGS. 10a to 10d show an example of a process for forming fine lattice patterns on the lower substrate.

Firstly, as shown in FIG. 10a, a plate 310 made of glass, silicon or ceramics is provided. A layer 320 of photoresist is formed on the plate, for example, by a spin coating. The plate 310 is used as a mold for molding a lower substrate.

Then, the layer of photoresist is patterned by exposure and developing processes, so that a mask pattern 320 having fine lattice patterns is formed on the plate.

Then, the plate 310 is etched by using the mask pattern 320 as an etching mask and removed by a strip process, resulting in a mold 310 having fine lattice patterns formed as shown in FIG. 10b.

As shown in FIG. 10c, melted state of plastics 200 which is heated to a predetermined temperature is poured in the mold 310. Then, the melted plastics 200 is cooled and cured in the mold 310.

After the plastics 200 is cured in the mold 310, the mold 310 is separated from the cured plastics 200, so that a lower substrate 200 having fine lattice patterns 210 formed is manufactured, as shown in FIG. 10d.

With the above method, the plate 310 itself is used as a mold. However, after an oxide, nitride or metal layer is additionally deposited on the plate 310 as a mold-forming layer, the mold-forming layer is formed with fine lattice patterns and can then be used as a mold.

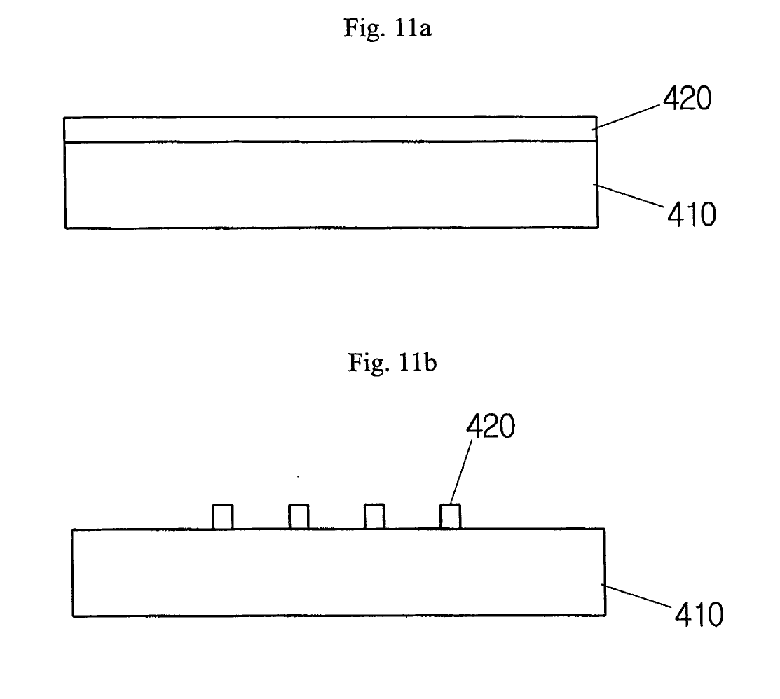

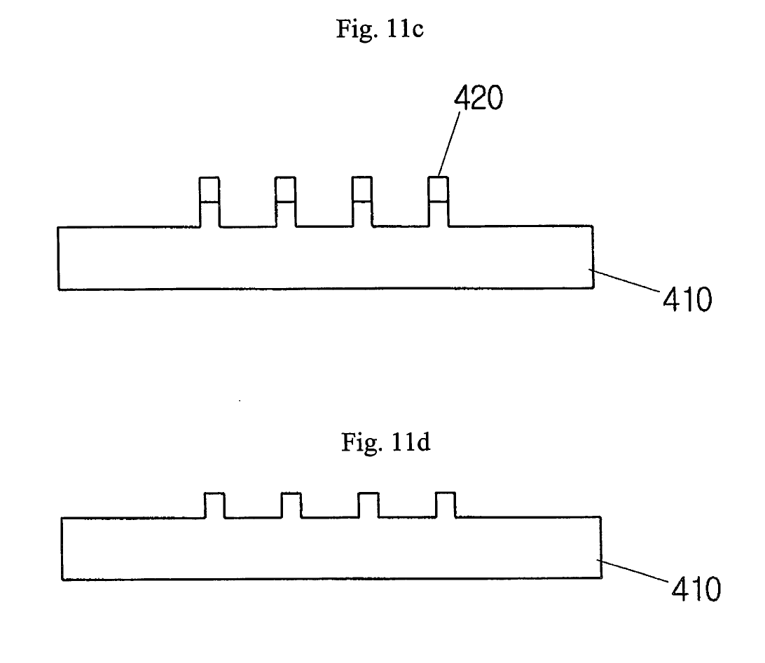

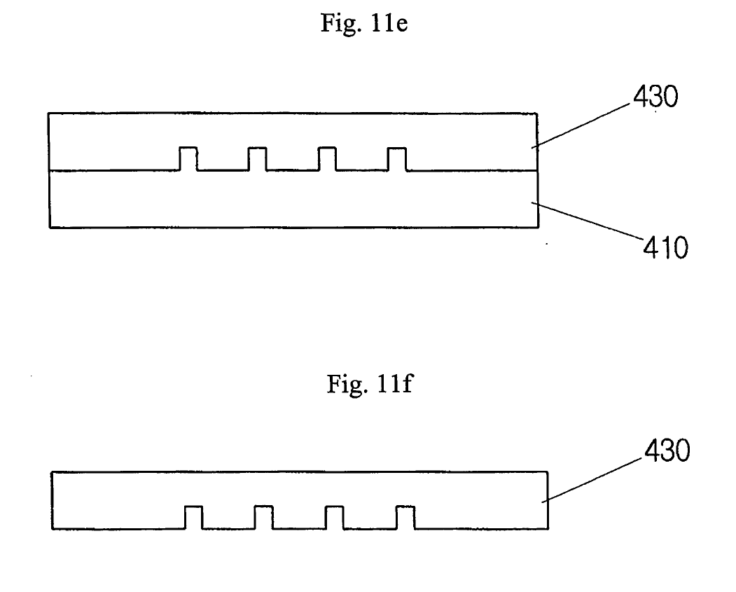

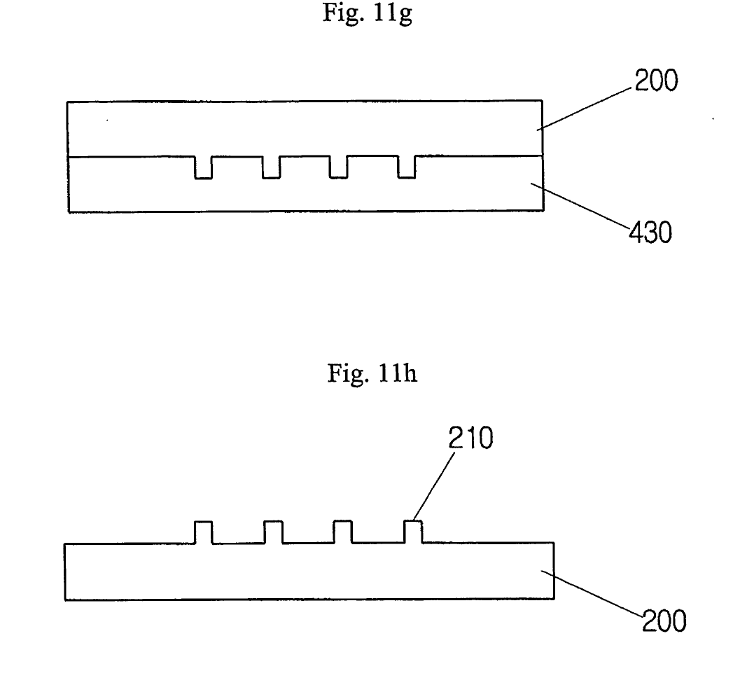

FIGS. 11a to 11h show sectional views for illustrating another example of a process forming fine lattice patterns on the lower substrate. In this example, contrary to the process shown in FIGS. 10a to 10d, a master for forming a mold is separately manufactured.

Firstly, as shown in FIG. 11a, a layer 420 of photoresist is formed on a plate 410 of glass, silicon or ceramics, which is used as a master, by a spin coating method, for example.

After that, as shown in FIG. 11b, the layer 420 of photoresist is patterned by exposure and developing processes.

Then, as shown in FIG. 11c, the plate 410 is etched by using the patterns 420 of the photoresist as an etching mask.

After that, as shown in FIG. 11d, the mask 420 is removed by a strip process, so that a master 410 having fine lattice patterns formed is provided.

Then, as shown in FIG. 11e, a Ni-layer 430 is formed on the master 410 by an electroless plating or electrolysis plating method. After that, the master 410 is removed, so that a mold 430 made of Ni is provided as shown in FIG. 11f. At this time, just before the plating step, the master is preferably surface-treated by a sputtering, vacuum vapor-deposition or non-electrolytic plating process so that the master 410 is electrically conducted.

Then, as shown in FIGS. 11g and 11h, a lower substrate 200 having fine lattice patterns 210 formed can be manufactured by a molding process using the mold 430.

Preferably, the upper or lower substrate made as described above is passed through an additional process such as a hydrophilic treatment or reactive group introduction. When the device of the invention is treated with oxygen-plasma, etc. to make the device hydrophilic, aqueous liquid such as blood can flow well and uniformly spread on the surface thereof. In addition, in order to introduce desired reactive group, for example, amine group, the device can be treated with plasma of the amine group or other chemical method (surface modification). Like this, when the device according to the invention is surface-treated, its performance is further improved.

### INDUSTRIAL APPLICABILITY

As described above, since the device of the invention is integratedly made by bonding the upper and lower substrates, a covering process as the prior device for counting cells is not required. Accordingly, since it is easy to fill the fill chamber with the sample by dropping the sample into the injection hole, the device can be more easily used than that of the prior art. Also, since the cost of manufacturing the device for counting cells is greatly decreased, the device can be disposably and easily used.

While the invention has been shown and described with reference to certain preferred embodiments thereof, it will be understood by those skilled in the art that various changes in form and details may be made therein without departing from the spirit and scope of the invention as defined by the appended claims.

1. A device for counting fine particles comprising,
a transparent lower substrate having fine lattice patterns for counting the fine particles formed on an upper surface thereof; and a transparent upper substrate stacked on the lower substrate, wherein the upper substrate comprises a fill chamber having a predetermined height from a bottom surface of the upper substrate and forming a space for filling a sample including the fine particles on the fine lattice patterns and an injecting hole for the sample communicated with the fill chamber.

2. The device according to claim 1, wherein the upper substrate further comprises a discharge hole communicated with the fill chamber for discharging the sample or an air bubble from the fill chamber.

3. The device according to claim 1, wherein the upper and lower substrates are bonded and thus form an integrated body.

4. The device according to claim 3, wherein the upper and lower substrates are bonded by a heating, an adhesive, a coating, a pressurization, a vibration or an ultrasonic bonding.

5. The device according to claim 1, wherein the fill chamber is formed with a height of 50-200 μm.

6. The device according to claim 1, wherein an area of the fill chamber in the upper and lower substrates is transparent and the fine lattice patterns are formed in a predetermined place of the area in which the fill chamber is formed on the lower substrate.

7. The device according to claim 1, wherein an indicative member is formed on the upper substrate for indicating a position of the fine lattice patterns.

8. The device according to claim 1, wherein the upper or lower substrate is made of plastics.

9. The device according to claim 1, wherein the fine particles are blood cells or bacteria.

10. A manufacturing method of a device for counting fine particles comprising steps of;
forming fine lattice patterns on a predetermined place of a lower substrate;
forming a fill chamber having a predetermined height for filling a sample including the fine particles, an injecting hole and a discharge hole communicated with the fill chamber in an upper substrate; and
bonding the upper and lower substrates.

11. The method according to claim 10, wherein the step of forming fine lattice patterns on a predetermined place of the lower substrate comprising steps of:
forming a photoresist layer on a plate;
forming a mask pattern having fine lattice patterns on the plate by patterning the photoresist layer;
etching the plate by using the mask pattern as an etching mask;
removing the mask pattern to obtain the plate as a mold wherein the fine lattice patterns are formed;
pouring melted plastics on the mold, and then cooling and curing the plastics on the mold; and
removing the mold to obtain the plastics as the lower substrate wherein the fine lattice patterns are formed.

12. The method according to claim 10, wherein the step of forming fine lattice patterns on a predetermined place of the lower substrate comprising steps of:
forming a photoresist layer on a plate;
forming a mask pattern having fine lattice patterns on the plate by patterning the photoresist layer;
etching the plate by using the mask pattern as an etching mask;
removing the mask pattern to obtain the plate as a master wherein the fine lattice patterns are formed;
forming Ni layer on the master by electroless plating or electrolysis plating;
removing the master to obtain the Ni layer as a mold wherein the fine lattice patterns are formed;
pouring melted plastics on the mold, and then cooling and curing the plastics on the mold; and
removing the mold to obtain the plastics as the lower substrate wherein fine lattice patterns are formed.
